(19) United States
(12) Patent Application Publication
(10) Pub. No.: US 2002/0094350 A1
(43) Pub. Date: Jul. 18, 2002

(54) Compositions containing an active fraction isolated from scutellariae barbatae and methods of use

(76) Inventor: Kin-Ping Wong, Fresno, CA (US)

Correspondence Address: McCutchen Doyle Brown & Enersen LLP, Three Embarcadero Center, Suite 1800, San Francisco, CA, 94111-4067, US

(21) Appl. No.: 10/016,977

(22) Filed: Dec. 12, 2001

Related U.S. Application Data
(60) Provisional application No. 60/254,986, filed on Dec. 12, 2000.

(51) Int. Cl. A61K 35/78

(52) U.S. Cl. 424/741

(57) ABSTRACT

This invention provides processes for isolating pharmaceutically active fractions from the mushroom Scutellariae barbatae designated herein as ESBa, ESBb and ASB03. This invention provides a method for inhibiting the growth of endothelial cells by delivering to the cells a growth inhibitory amount of a fraction of this invention. This invention also provides a method of inhibiting vascularization in a tissue by delivering to the tissue an anti-vascularization amount of a fraction of this invention. Methods of treating various diseases, including cancer, are also provided herein.

### CROSS-REFERENCE TO RELATED APPLICATIONS

This application claims the benefit under 35 U.S.C. §119(e) of U.S. Provisional Application No. 60/254,986, filed Dec. 12, 2000, the contents of which are hereby incorporated by reference into the present disclosure.

### FIELD OF THE INVENTION

The present invention is in the field of pharmaceuticals. In particular, it is related to the field of anti-angiogenic pharmaceuticals for the prevention and treatment of disease.

### BACKGROUND

Angiogenesis is the process through which new vascular structures arise by outgrowth from pre-existing capillaries. In this process, endothelial cells become detached from the basement membrane as this support is degraded by proteolytic enzymes. These cells then migrate out from the parent vessel, divide, and form into a newly differentiated vascular structure (Risau, (1997) Nature 386:671-674; Wilting et al., (1995) Cell. Mol. Biol. Res. 41(4):219-232). A variety of different biological factors have been found to function in controlling blood vessel formation (Bussolino et al., (1997) Trends in Biochem Sci 22(7):251-256; Folkman and D'Amore, (1996) Cell 87:1153-1155). These include proteins with diverse functions such as growth factors, cell surface receptors, proteases, protease inhibitors, and extracellular matrix proteins (Achen and Stacker, (1998) Int. J. Exp. Pathol. 79:255-265; Devalaraja and Richmond, (1999) Trends in Pharmacol. Sci. 20(4):151-156; Hanahan, (1997) Science 277:48-50; Maisonpierre et al, (1997) Science 277:55-60; Suri et al, (1996) Cell 87:1171-1180; Sato et al, (1995) Nature 376:70-74; Mignatti and Rifkin, (1996) Enzyme Protein 49:117-137; Pintucci et al., (1996) Semin Thromb Hemost 22(6)517-524; Vernon and Sage, (1995) Am. J. Pathol. 147(4):873-883; Brooks et al., (1994) Science 264:569-571; Koch et al., (1995) Nature 376:517-519). The complexity of the angiogenic process and the diversity of the factors that control its progression provide a useful array of points for therapeutic intervention to control vascular formation in vivo.

Angiogenesis normally occurs in a carefully controlled manner during embryonic development, during growth, and in special cases such as wound healing and the female reproductive cycle (Wilting and Christ, (1996) Naturwissenschaften 83:153-164; Goodger and Rogers, (1995) Microcirculation 2:329-343; Augustin et al., (1995) Am. J. Pathol. 147(2):339-351). Some of the important steps in the process of angiogenesis are: 1) growth factor (i.e. vascular endothelial growth factor, VEGF) signaling; 2) matrix metalloproteinases (MMP) and VEGF receptor interaction; 3) endothelial cell migration to site of growth factor signaling; and 4) endothelial cell tubule formation. Pathological angiogenesis plays a central role in a number of human diseases including tumor growth and metastatic cancer, diabetic retinopathy, rheumatoid arthritis, and other inflammatory diseases such as psoriasis (Folkman, (1995) Nature Med. 1(1):27-31; Polverini, (1995) Rheumatology 38(2):103-112; Healy et al., (1998) Hum. Reprod. Update 4(5):736-396). In these cases, progression of disease is driven by persistent unregulated angiogenesis. For example, in rheumatoid arthritis, new capillary blood vessels invade the joints and destroy the cartilage. In diabetic retinopathy, capillaries in the retina invade the vitreous, bleed and cause blindness. In diabetic retinopathy, capillaries in the retina invade the vitreous, bleed and cause blindness. Significantly, tumor growth and metastisis are angiogenesis dependent. Most primary solid tumors go through a prolonged avascular state during which growth is limited to approximately 1-2 mm in diameter. Up to this size, tumor cells can obtain the necessary oxygen and nutrient supply by passive diffusion. These microscopic tumor masses can eventually switch on angiogenesis and recruit surrounding blood vessels to begin sprouting capillaries that vascularize the tumor mass, providing the potential for continuing expansion of the tumor and metastasis of malignant cells to distant locations. Although significant progress has been made in understanding the biological events that occur during pathological angiogenesis, there are presently no effective pharmaceutical compounds that are useful for controlling angiogensis in vivo. Thus, effective therapies capable of controlling angiogenesis have the potential to alleviate a significant number of human diseases.

Traditionally, pharmaceutical compounds have been developed by screening synthetic chemical compounds for desirable pharmaceutical properties and then testing them for toxicity and effectiveness in vivo. Compounds selected this way frequently have toxic side effects in vivo and this approach has not been successful in developing effective angiogensis inhibitors for disease therapy. More recently, techniques of molecular biology have been applied to develop angiogenesis inhibitors. Protein inhibitors of angiogenesis such as angiostatin (O'Reilly et al., (1994) Cell 79(2):315-328) and endostatin (O'Reilly et al., (1997) Cell 88(2):277-285), that control vascular formation in experimental models, have been discovered. Nevertheless, such protein therapeutics are expensive to produce and have been found to be difficult to formulate and deliver in subjects. At present, protein angiogensis inhibitors have yet to be developed into therapeutic pharmaceuticals for disease patients. Thus, there exists a need for therapeutic compounds that can be safely administered to a patient and be effective at inhibiting the pathological growth of vascular endothelial cells. The present invention provides compositions and methods that are useful for this purpose and provides related advantages as well.

### DISCLOSURE OF THE INVENTION

This invention provides processes for extracting pharmaceutically active fractions (also termed “extract”, “compound” or “drug”) from the Scutellariae barbatae. In one aspect, the process is extracting from a hot (at least about 60° C. and more preferably, about 100° C.) tea of Scutellariae barbatae a fraction soluble in an organic solvent having an optical absorbance between about 200 nm and about 400 nm. One means to obtain this fraction is by steeping an effective amount of Scutellariae barbatae in an effective amount of hot water to obtain a liquid extract and then filtering the extract to obtain a filtrate. This is then extracted with an effective amount of an organic solvent. The solvent is removed under nitrogen and the pellet is resuspended in water. The suspension is concentrated and the pharmaceutically active fraction is separated by chromatography. The pharmaceutically active fractions obtainable by these methods, i.e., ESBa, ESBb and ASBO3, are also provided herein.

This invention provides methods for inhibiting the growth of endothelial cells by delivering to the cells a growth inhibitory amount of a fraction of the invention. There are useful to inhibit vascularization in a tissue by delivering to the tissue an anti-vascularization amount of a fraction. Methods of treating various diseases, including cancer, are also provided herein.

### BRIEF DESCRIPTION OF THE FIGURES

FIGS. 1 through 5 depict exemplary processes of this invention. However, it is to be understood, although not always explicitly stated that the reagents described are merely exemplary and that equivalents of such are well known in the art. The following are examples and equivalents thereof are within the scope of this invention.

FIG. 1 depicts procedures for isolating the active fractions designated ESBa and ESBb that are useful as food and health supplements. ESBa is the first crude extract in the process from the initial extraction from the plant to after the first lyophilization step. This extract contains anti-angiogenic properties. The second crude extract, ESBb, is obtained by a continuation of the process. After the first lyophilization, purification is continued by doing first a 95% ethanol wash, and then an absolute methanol extraction. This process has been found to further isolate the active fraction found in ESBa. The extract from this ethanol wash/methanol extraction is dissolved in water and then lyophilized, resulting in a more concentrated and purer active extract from Scutellareia barbatae which is called ESBb. This more concentrated and purer extract is further purified by first dissolving in water, then adsorbing to a C-18 mini-column. After washing with water, the adsorbed active component is desorbed from the C-18 mini-column by a 25% methanol wash. The purified ASBO3 is obtained from elution on a HPLC C-18 hydrophobic column with a 0 to 100% methanol gradient. All fractions, pellets, and supernatants are assayed for anti-angiogenic activity using the CPAE assay after each step to insure that no anti-angiogenic is lost or misplaced.

FIG. 2 depicts the HPLC elution from a C-18 hydrophobic chromatograph column of a methanol extract of Scutellariae barbatae.

FIG. 3 depicts an alternative procedure for the extraction and purification of ASBO3 from Scutellariae barbatae.

FIG. 4 is a graph depicting the results of analysis of the anti-angiogenic activity of ASB03 using a G-25 column.

FIG. 5 (panels A, B, and C) depict UV spectrophotometric analysis of the active fractions of ASB03 from HPLC C-18 chromatography extract isolated from Scutellariae barbatae.

### MODES FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The practice of the present invention employs, unless otherwise indicated, conventional techniques of molecular biology (including recombinant techniques), microbiology, cell biology, organic chemistry, biochemistry and immunology, which are within the skill of the art. Such techniques are explained fully in the literature.

Definitions

As used herein, certain terms may have the following defined meanings.

As used in the specification and claims, the singular form “a,”“an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are well known in the art.

The term “isolated” means separated from constituents, cellular and otherwise, in which the compound is normally associated with in nature.

A “subject” or “host” is a vertebrate, preferably an animal or mammal, more preferably a human patient. Mammals include, but are not limited to, murines, simians, human patients, farm animals, sport animals, and pets.

The terms “cancer,”“neoplasm,” and “tumor,” used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition.

As used herein, “inhibit” means to stop, delay or slow the growth, proliferation or cell division of endothelial cells or the formation of blood vessels in tissue. Methods to monitor inhibition include, but are not limited to endothelial cell proliferation assays, measurement of the volume of a vascular bed by determination of blood content and quantitative determination of the density of vascular structures. When the culture is a mixture of cells, neovascularization is monitored by quantitative measurement of cells expressing endothelial cell specific markers such as angiogenic factors, proteolytic enzymes and endothelial cell specific cell adhesion molecules.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

An “effective amount” is an amount sufficient to effect beneficial or desired results. This amount may be the same or different from a prophylatically effective amount, which is an amount necessary to prevent onset of disease or disease symptoms. An effective amount can be administered in one or more administrations, applications or dosages.

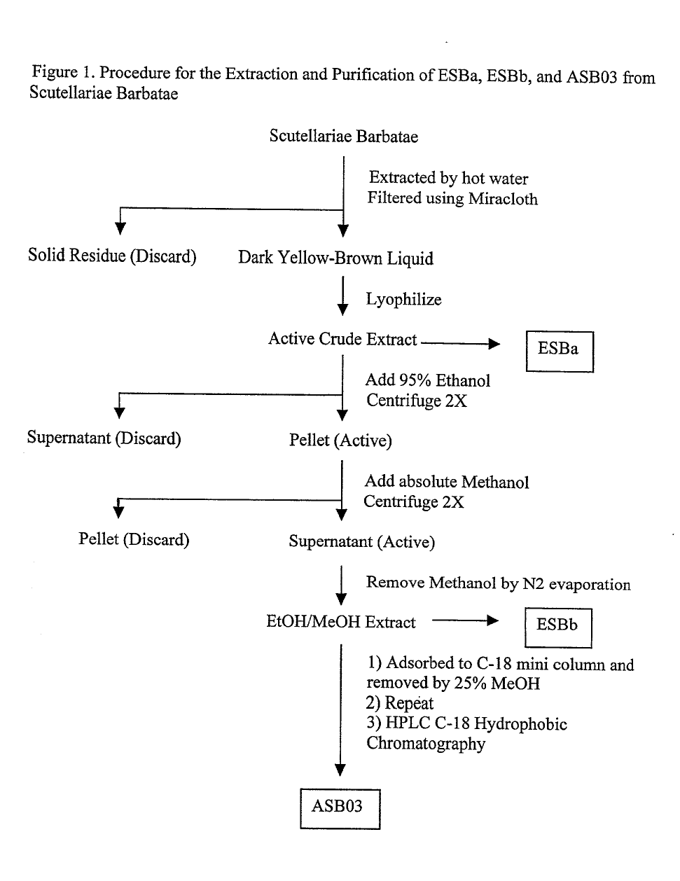

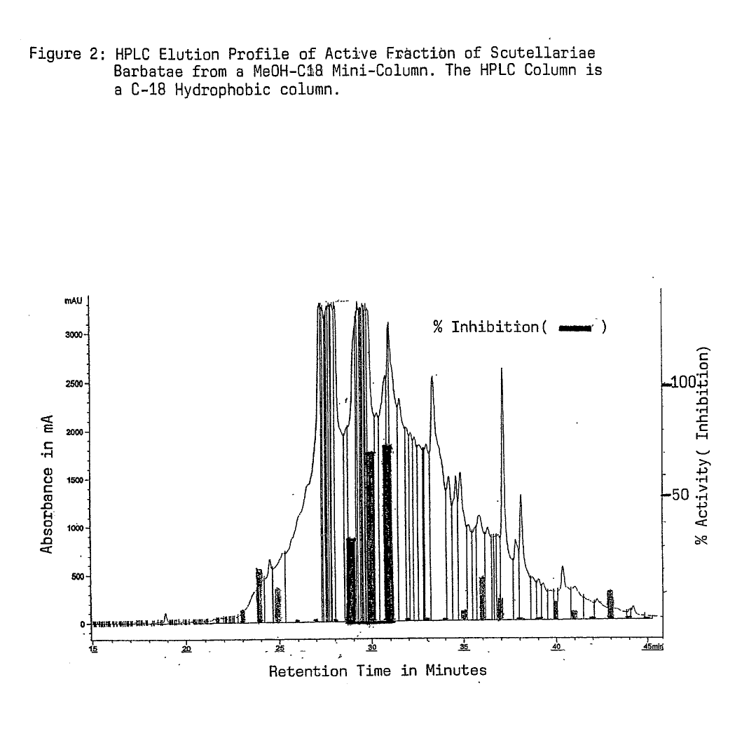

Applicant has identified process for extracting pharmaceutically active fractions from the Scutellariae barbatae. In one aspect, the process is extracting from a hot (about 100° C.) tea of Scutellariae barbatae a fraction soluble in an organic solvent wherein the fraction has an optical absorbance between about 200 nm to about 500 nm, and more preferably between about 200 nm and about 400 nm. One means to obtain this fraction is by steeping an effective amount of Scutellariae barbatae in an effective amount of hot water to obtain a liquid extract and then filtering the extract to obtain a filtrate. This fraction is designated ESBa and contains anti-angiogenic properties (See FIG. 1 and 2). It can be concentrated and ingested as a food or health supplement. (See FIG. 1).

This is then extracted with an effective amount of an organic solvent. In one aspect also shown in FIG. 1, the concentrate is first washed with ethanol (95%) and then extracted with absolute methanol and concentrated to isolated ESBb that also possesses anti-angiogenic properties.

In an alternative embodiment, the fraction designated ESBa is extracted with an organic solvent (See FIG. 1). The solvent is removed under nitrogen and the pellet is resuspended in water. The suspension is concentrated and the pharmaceutically active fraction is separated by chromatography.

The inventor has also discovered that the extracts ESBa, ESBb and the active component ASB03, inhibit endothelial cell growth and possesses anti-angiogenic properties. In accordance with these findings, this invention provides methods for inhibiting the growth of endothelial cells by delivering to the cells a growth inhibitory amount of a fraction. This invention also provides methods of inhibiting vascularization in a tissue by delivering to the tissue an anti-vascularization amount of a fraction of this invention.

This method can be practiced in vitro or in vivo. When practiced in vitro, endothelial cells or vascularized tissue are cultured under conditions well known to skill in the art, e.g., as exemplified below. The cells and/or tissue can be from an established cell line or cultured from a biopsy sample obtained from a subject. The fraction is then directly added to the culture medium or delivered as a component of a pharmaceutical composition.

Not every therapy is effective for each individual and therefore, an in vitro assay to gauge efficacy for each patient would be advantageous. The present method provides these means to determine whether compositions or therapies will treat a subject's specific disease related to pathological proliferation of endothelial cells or vascularization. For example, a tissue biopsy is isolated from the patient and contacted with an effective amount of a pharmaceutical composition or therapy as defined herein and under conditions effective for growth and proliferation of the cells. Inhibition of growth of the pathological cells as determined by conventional procedures, e.g., the CPAE assay described herein, indicates that the inventive compositions and/or therapies may effectively treat the patient.

This invention also provides a method of treating a disorder associated with pathological neovascularization in a subject by administering to the subject a therapeutically effective amount or a growth inhibitory amount of a fraction of this invention, or a pharmaceutically acceptable derivative, salt or prodrug thereof. As used in this context, to “treat” means to alleviate the symptoms associated with pathological neovascularization as well as the reduction of neovascularization. Such conditions include, but are not limited to arthritic conditions, neovascular-based dermatological conditions, diabetic retinopathy, Karposi's Sarcoma, age-related macular degeneration, restenosis, telangectasia, glaucoma, keloids, corneal graft rejection, wound granularization, angiofibroma, Osler-Webber Syndrome, myocardial angiogenesis, and scleroderma. Exemplary arthritic conditions are selected from the group consisting of psoriatic arthritis, rheumatoid arthritis and osteoarthritis. For the treatment of cancers and solid tumors, to “treat” includes inhibition of the growth of blood vessels resulting in a lack of nutrients for the tumors and/or cancer cells needed by the tumor for its growth. Tumors and growths will decrease in size and possibly disappear. Administration for the treatment of arthritic conditions will result in decreased blood vessel formation in cartilage, specifically joints, resulting in increased mobility and flexibility in these regions. For the treatment of psoriasis, administration will reduce dermatological symptoms such as scabbing, flaking and visible blood vessels under the surface of the skin. In diabetic retinopathy, administration of a fraction of this invention will reduce the formation of extraneous blood vessels in the retina, resulting in unobstructed vision. In the treatment of Kaposi's sarcoma, administration of a fraction of this invention will inhibit the growth and/or further formation of blood vessels, thereby inhibiting the formation of lesions and/or tumors that arise.

When a fraction is administered to a subject such as a mouse, a rat or a human patient, the fraction can be added to a pharmaceutically acceptable carrier and systemically, orally, transdermally or topically administered to the subject. Therapeutic amounts can be empirically determined and will vary with the pathology being treated, the subject being treated and the toxicity of the form of the active fraction used in the therapeutic method. An active fraction in its various forms can be delivered orally, intravenously, intraperitoneally, or transdermally. When delivered to an animal, the method is useful to further confirm efficacy of the active fraction.

As an example of an animal model, groups of nude mice (Balb/c NCR nu/nu female, Simonsen, Gilroy, Calif.) are each subcutaneously inoculated with about 10⁵ to about 10⁹ pathological cells as defined herein. When the graft is established, the fraction is administered, for example, by subcutaneous injection around the graft. Measurements to determine reduction of graft size are made in two dimensions using venier calipers twice a week.

The MRL/lpr mice (MRL/MpJ-Fas^{lpr}) from Jackson Labs (Maine) are useful to test or monitor efficacy in arthritic conditions. A positive therapeutic benefit includes reduced swelling of the joints and hindlegs of animals and reduced cartilage degradation which can be monitored by X-ray.

Administration in vivo can be effected in one dose, multiple doses, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering an active fraction is described herein.

The compositions and pharmaceutical formulations of the present invention can be used in the manufacture of medicaments and for the treatment of humans and other animals by administration in accordance with conventional procedures, such as an active ingredient in pharmaceutical compositions.

The fractions or compounds can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to the active fraction of the present invention, the pharmaceutical compositions can also contain other pharmaceutically active agents.

An active fraction can be administered for therapy by any suitable route including oral, rectal, nasal, topical (including transdermal, aerosol, buccal and sublingual), vaginal, parental (including subcutaneous, intramuscular, intravenous and intradermal) and pulmonary. It will also be appreciated that the preferred route will vary with the condition and age of the recipient, and the disease being treated.

It will be appreciated that appropriate dosages of the active fraction depend on the type and severity and stage of the disease and can vary from patient to patient. The optimal dosage involves the balancing of the level of therapeutic benefit against any risk or deleterious side effects of the treatments of the present invention.

Ideally, the extract (“drug”) or composition containing it should be administered to achieve peak concentrations of the active compound at sites of disease. This may be achieved, for example, by the intravenous injection of the drug, optionally in saline, or orally administered, for example, as a tablet, capsule or syrup containing the active ingredient. Desirable blood levels of the drug may be maintained by a continuous infusion to provide a therapeutic amount of the active ingredient within disease tissue. The use of operative combinations is contemplated to provide therapeutic combinations requiring a lower total dosage of each component agent than may be required when each individual therapeutic compound or drug is used alone, thereby reducing adverse side effects.

While it is possible for an active fraction or compound to be administered alone, it is preferable to present it as a pharmaceutical formulation comprising at least one active ingredient, as defined above, together with one or more pharmaceutically acceptable carriers therefor and optionally other therapeutic agents. Each carrier must be “acceptable” in the sense of being compatible with the other ingredients of the formulation and not injurious to the patient.

Formulations include those suitable for oral, rectal, nasal, topical (including transdermal, buccal and sublingual), vaginal, parenteral (including subcutaneous, intramuscular, intravenous and intraderinal) and pulmonary administration. The formulations may conveniently be presented in unit dosage form and may be prepared by any methods well known in the art of pharmacy. Such methods include the step of bringing into association the active ingredient with the carrier that constitutes one or more accessory ingredients. In general, the formulations are prepared by uniformly and intimately bringing into association the active ingredient with liquid carriers or finely divided solid carriers or both, and then if necessary shaping the product.

Formulations of the present invention suitable for oral administration may be presented as discrete units such as capsules, cachets or tablets, each containing a predetermined amount of the active ingredient; as a powder or granules; as a solution or suspension in an aqueous or non-aqueous liquid; or as an oil-in-water liquid emulsion or a water-in-oil liquid emulsion. The active ingredient may also be presented a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention may be formulated as an ointment, cream, suspension, lotion, powder, solution, paste, gel, spray, aerosol or oil. Alternatively, a formulation may comprise a patch or a dressing such as a bandage or adhesive plaster impregnated with active ingredients and optionally one or more excipients or diluents.

For diseases of the eye or other external tissues, e.g., mouth and skin, the formulations are preferably applied as a topical ointment or cream containing the active ingredient. When formulated in an ointment, the drug may be employed with either a paraffinic or a water-miscible ointment base. Alternatively, the drug ingredients may be formulated in a cream with an oil-in-water cream base.

If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound that enhances absorption or penetration of an active fraction through the skin or other affected areas. Examples of such dermal penetration enhancers include dimethylsulfoxide and related analogues.

The oily phase of the emulsions of this invention may be constituted from known ingredients in any known manner. While this phase may comprise merely an emulsifier (otherwise known as an emulgent) it desirably comprises a mixture of at lease one emulsifier with a fat or an oil or with both a fat and an oil. Preferably, a hydrophilic emulsifier is included together with a lipophilic emulsifier that acts as a stabilizer. It is also preferred to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulphate.

The choice of suitable oils or fats for the formulation is based on achieving the desired cosmetic properties, since the solubility of the active compound in most oils likely to be used in pharmaceutical emulsion formulations is very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient, such carriers as are known in the art to be appropriate.

Formulations suitable for nasal administration, wherein the carrier is a solid, include a coarse powder having a particle size, for example, in the range of about 20 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active ingredient.

Formulations suitable for parenteral administration include aqueous and nonaqueous isotonic sterile injection solutions which may contain anti-oxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily subdose, as herein above recited, or an appropriate fraction thereof, of a drug ingredient. They may also contain additional active agents, e.g., anti-tumor, anti-cancer, anti-angiogenic or immune enhancers.

It should be understood that in addition to the ingredients particularly mentioned above, the formulations of this invention may include other agents conventional in the art having regard to the type of formulation in question, for example, those suitable of oral administration may include such further agents as sweeteners, thickeners and flavoring agents.

The extract (“drug”) or compositions of the same may also be presented for the use in the form of veterinary formulations, which may be prepared, for example, by methods that are conventional in the art.

This invention further provides a method for screening for a therapeutic agent for inhibiting neovascularization or endothelial cell growth. The screen comprises:

(a) contacting the agent with a suitable cell or tissue sample;

(b) contacting a separate sample of the suitable cell or tissue with a therapeutically effective amount of the extract of this invention or a pharmaceutically acceptable composition containing the extract; and

(c) comparing the growth of the sample of step (a) with the growth of the sample of step (b), and wherein any agent of step (a) that inhibits the growth to the same or similar extent as the sample of step (b) is a therapeutic agent for inhibiting neovascularization or the growth of endothelial cells.

As used herein, a suitable sample intends any sample that contains endothelial cells. The method can be practiced in vitro or in vivo as described herein.

A kit for treating a disorder associated with pathological neovascularization in a subject, also is provided by this invention. The kit includes a therapeutically effective amount of the extract and instructions for use. The kit is useful to treat disorders selected from the group consisting of arthritic conditions, neovascular-based dermatological conditions, diabetic retinopathy, Karposi's Sarcoma, age-related macular degeneration, restenosis, telangectasia, glaucoma, keloids, corneal graft rejection, wound granularization, angiofibroma, Osler-Webber Syndrome, myocardial angiogenesis, scleroderma, psoriatic arthritis, rheumatoid arthritis and osteoarthritis.

The following examples are intended to illustrate, but not limit the invention.

### EXAMPLES

### Example 1

Isolation and Purification of Active Fractions

This invention provides several embodiments for preparing a biologically active fraction from a composition containing Scutellariae barbatae. In one aspect, (depicted in FIG. 1) the process comprises extracting a fraction soluble in an organic solvent to obtain an active fraction having an optical absorbance of between about 200 mn to about 500 nm, and more preferably between about 200 nm and about 400 nm. In a further aspect, the fraction has an absorbance between about 260 nm and 330 nm.

In one embodiment, the process requires extracting from a combination of 10 grams of dried Scutellariae barbatae and about 300 ml of hot (at least about 60° C. and more preferably at least about 100° C.) doubly distilled water. It is steeped for about 5 minutes to 30 minutes and then filtered through a suitable filter material having a pore size about 22 to 25 microns. Suitable filters are autoclavable and sterilizable and highly resistant to numerous chemical solvents. One suitable filter is commercially available Miracloth. This extract (ESBa, see FIG. 1) has been shown to possess anti-angiogenic properties. It can be used as is or further processed for use as a food or health supplement.

The “tea” is then extracted at least 3 times with an organic solvent such as ethanol or methanol, or other similar organic solvents and then extracted between 2 and 5, and more preferably about 4 times with methanol. The extract (ESBb, see FIG. 1) also possesses anti-angiogenic activity. It can be used as is or further processed for use as a food or health supplement.

ESBb can be further processed by concentration, e.g., dissolve in water and lyophilization. The lyophylate is then separated into the active fraction ASB03 (See FIG. 1) using column chromatography to obtain a fraction having an optical absorbance at from about 200 to about 500 nm, or more preferably between about 200 and about 400 nm and most preferably between about 300 nm and about 400 mn.

In an alternative embodiment, the steps for obtaining an active fraction are:

a) steeping for at least 5 minutes an effective amount of Scutellariae barbatae in hot doubly distilled water.

b) the liquid is then centrifuged (at 2000 rpm) for about 10 minutes and filtered with Watman filter paper. The precipitate is discarded.

c) the crude extract is lyophilized overnight to obtain ESBa.

d) after washing with 95% ethanol and centrifugation, the pellet is extracted with an effective amount of an organic solvent to obtain a purer extract.

e) the organic solvent (e.g., methanol) is evaporated with nitrogen gas.

f) the dried suspension is mixed with at least about 12 to about 18 ml of water and lyophilized overnight to obtain ESBb.

g) it can be resuspended in a pharmaceutically acceptable carrier such as doubly distilled water.

h) the solution is run over a column for chromatographic separation.

AC-18 column is one such column. A 25% methanol solution is used to elute out the absorbed or active fractions.

i) the methanol is evaporated with a stream of nitrogen gas.

j) the extract is resuspended in water and lyophilized.

k) this is then fractionated using for example, column chromatography such as HPLC in a 0 to 100% gradient to obtain ASB03.

### Example 2

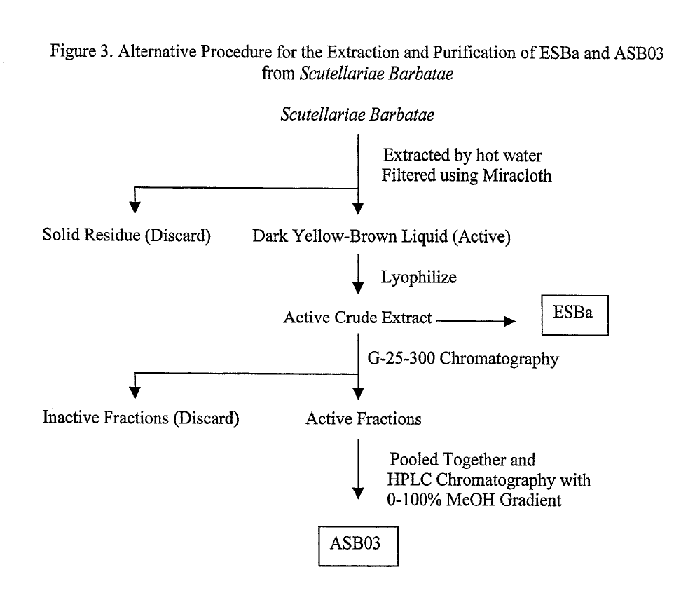

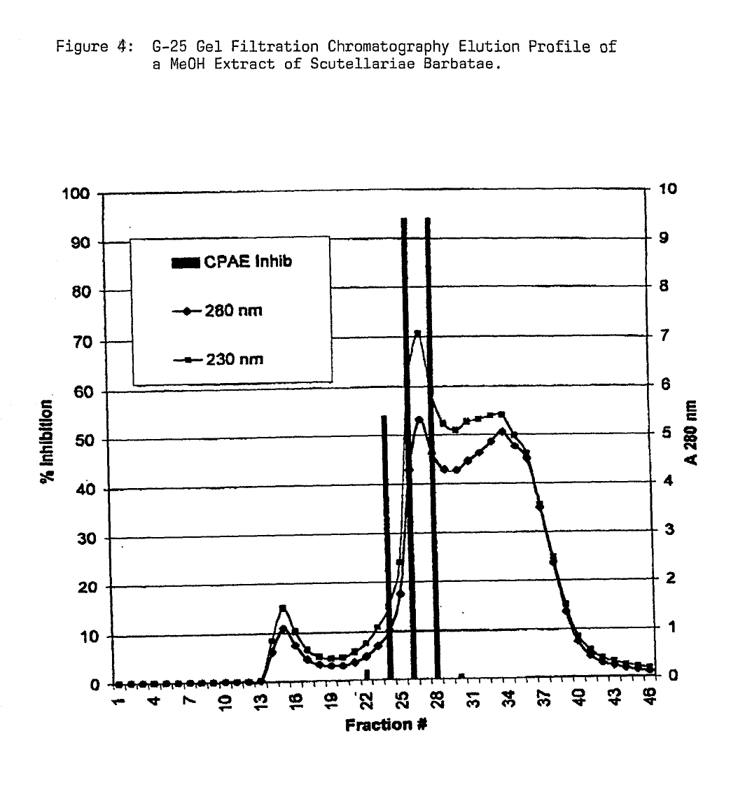

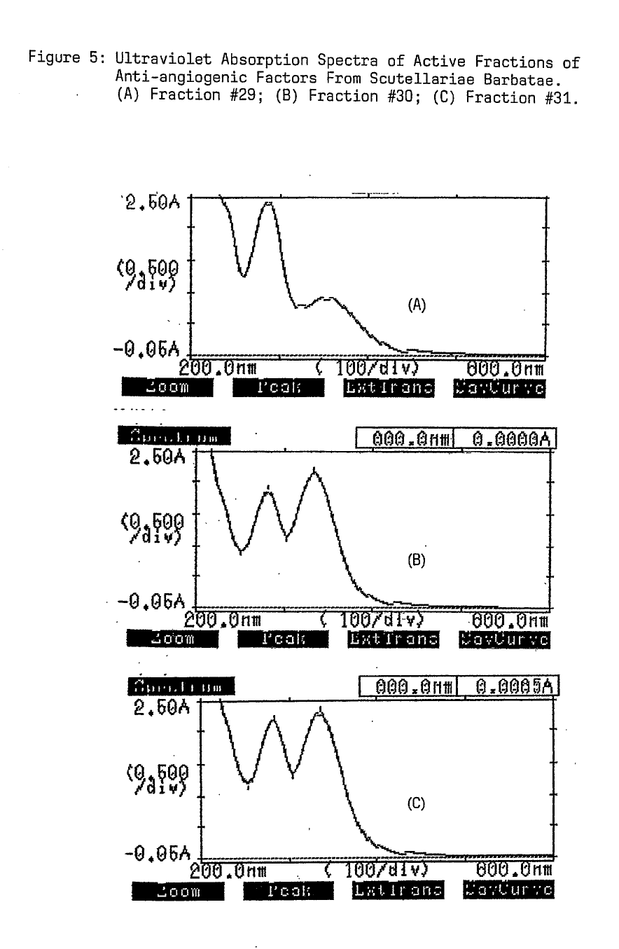

FIG. 3 shows an alternative procedure for isolating an active fraction designated ASB03. In this embodiment, a crude extract is obtained as described in Example 1, above, and then fractionated using a G-25-300 (25×40 cm) column. FIG. 4 depicts anti-angiogenic activity of the different fractions isolated off the G-25 column. Fractions were obtained by running the column at a flow rate of 2 ml/min. Fractions 22 through 30 were biologically active as determined using CPAE assay (described below).

### Example 3

Endothelial Cell Assays (“CPAE”)

The assays were carried out according to the procedures of Connally, et al. (1986) Anal. Biochem. 152:136-4 with modifications (Liang and Wong (1999) ANGIOGENESIS: FROM THE MOLECULAR TO INTEGRATIVE PHARMACOLOGY edited by Maradoudakis, Kluwer Academic/Plenum. Publishers, New York). Calf Pulmonary Arterial Endothelial (CPAE) cells are plated at 10,000 cells per well in 24 well culture plates. After growth incubation at 37° C., 5% CO₂ for about 60 hours, a dosage of the sample is added (about 50 μl to about 100 μl) to each sample well and re-incubated for 30 minutes. After incubation, cells are assayed visually under an inverted microscope to detect the presence of cells and through the use of the ECC assay. Both methods are used to detect the presence or absence of endothelial cells in each well. Control cells containing no sample were used and grew normally.

### Example 4

CAM Assay

The chorioallantoic membrane (CAM) assay (Nguyen, M., et al. (1994) Microvas. Res. 47:31-40) is used to determine the efficacy of an active fraction, extract or compound in an in vivo model. Fertilized chicken eggs are obtained from Kings Valley Farms (Kingsburg, Calif.) and incubated at 37° C. in a humidified chamber. Eggs are rotated 180 once daily for four days at which time windows are cut in the shells as follows: First, eggs are swabbed with 95% ethanol and a small hole placed in the blunt end of the egg which will collapse the air sack present there. The membrane falls away from the shell when a 1 cm² “window” is removed from the shell using a hacksaw (25 teeth/in). Sterilized Howard's Ringer solution is used to wash away any excess shell fragments. The eggs are then sealed with cellophane tape and returned to the incubator. After 4-7 days, the window is reopened and a sterile cover slip is placed to serve as the reference point. For testing, a hole is created with a sterile needle between blood vessels in the CAM and a small amount of sample was applied to the hole, which is then resealed. Lactose is used as the control. Observations are made daily for 4 days for any inhibition of blood vessel development on the CAM.

### Example 5

MMP Assay

P. C. Brooks, et. al. (1996) in “Localization of matrix metalloproteinase MMP-2 to the surface of invasive cells by interaction with integrin αvβ3,” Cell 85:683-93 describes an in vitro assay on matrix metalloproteinase and αv/β3 integrin interaction. The effects of the experimental sample on the MMP-2/αvβ3 integrin complex determines if the sample's mechanism of action involves any disruption of this segment of the angiogenic pathway. This involves testing if the experimental sample can inhibit the interaction of MMP-2 with the αvβ3 integrin. Initially, this is done via an ELISA using antibodies for MMP-2 and testing the binding of these antibodies to the sample. Further studies are pursued if a positive result occurs. TIMP-2 (Tissue Inhibitor of Matrix Metalloprotease-2), a known natural inhibitor of MMP-2, is used as the control.

### Example 6

Endothelial Cell Tubule/Cord Formation Assay

Matrigel (60 μl of 10 mg/ml; Collaborative Lab # 35423) is placed in each well of an ice-cold 96-well plate. The plate is allowed to sit at room temperature for 15 minutes then incubated at 37° C. for 30 minutes to permit the matrigel to polymerize. In the mean time, HUVEC are prepared in EGM-2 (Clonetic # CC3162) at a concentration of 2×10⁵ cells/ml. The test compound is prepared at 2× the desired concentration (5 concentration levels) in the same medium. Cells (500 μl) and 2× fraction or compound (500 μl) is mixed and 200μl of this suspension are placed in duplicate on the polymerized matrigel. After a 24 hour incubation, triplicate pictures are taken for each concentration using a Bioquant Image Analysis system. Drug effect (IC₅₀) is assessed compared to untreated controls by measuring the length of cords/tubules formed and number of junctions. TNP-470 (NSC 642492) and paclitaxel (NSC 125973) are used as reference compounds.

### Example 7

Endothelial Cell Migration Assay

Migration is assessed using the 48-well Boyden chamber and 8μm pore size collagen-coated (10 μg/ml rat tail collagen; Collaborative Laboratories) polycarbonate filters (Osmonics, Inc.). The bottom chamber wells receive 27-29 μl of DMEM medium alone (baseline) or medium containing chemo-attractant (bFGF, VEGF or Swiss 3T3 cell conditioned medium). The top chambers receive 45 μl of HUVEC cell suspension (1×10⁶ cells/ml) prepared in DMEM+1% BSA with or without the fraction or compound. After a 5 hour incubation at 37° C., the membrane is rinsed in PBS, fixed and stained in Diff-Quick solutions. The filter is placed on a glass slide with the migrated cells facing down and cells on top are removed using a Kimwipe. The testing is performed in 4-6 replicates and five fields are counted from each well. Negative unstimulated control values are subtracted from stimulated control and fraction or compound treated values and data is plotted as mean migrated cell ±S.D. IC₅₀ is calculated from the plotted data. TNP-470 (NSC 642492) and paclitaxel (NSC 125973) are used as reference compounds.

Although the foregoing invention has been described in some detail by way of illustration and example for purposes of clarity of understanding, it will be apparent to those skilled in the art that certain changes and modifications will be practiced. For example, as is apparent to those of skill in the art, the invention method can be combined with one or more known anti-tumor, anti-angiogenic or immune enhancing therapies and compositions, e.g., shark cartilage, tyrosphingosine or sphingosine. Therefore, the description and examples should not be construed as limiting the scope of the invention, which is delineated by the appended claims.

What is claimed is:

1. A process for preparing a biologically active fraction designated ASB03 from a composition of Scutellariae barbatae comprising extracting a fraction soluble in an organic solvent wherein said fraction has an optical absorbance between about 200 nm to about 500 nm.

2. The process of claim 1, comprising the steps of:
a) steeping an effective amount of Scutellariae barbatae in an effective amount of hot water to obtain a liquid extract;
b) filtering the extract to obtain a filtrate designated ESBa;
c) extracting the filtrate with an effective amount of an organic solvent to obtain ESBb;
d) concentrating the extract; and
e) isolating a fraction having an optical absorbance at about 200 nm to about 450 nm to isolate ASB03.

3. The process of claim 1, comprising the steps of:
a) obtaining a supernatant by extracting at least two times with ethanol from a liquid extract of Scutellariae barbatae;
b) obtaining a concentrated supernatant by extracting at least three times with methanol;
c) evaporating the methanol with nitrogen gas;
d) resuspending the extract in about 12 to about 18 ml of water and drying;
e) resuspending in a pharmaceutically acceptable carrier to form a solution;
f) running the solution over a C-18 mini-column;
g) washing the column with at least about 35 ml of water;
h) washing the column with at least 15 ml of 25% methanol;
i) evaporating the methanol with nitrogen gas;
j) resuspending the extract in a liquid carrier; and
k) obtaining the active fraction by a C-18 hydrophobic HPLC chromatography to obtain a biologically active extract designated ASB03 having an optical absorbance from about 200 nm to about 500 nm.

4. A biologically active extract obtained by the process of claim 1.

5. A composition comprising the extract of claim 4 and a pharmaceutically acceptable carrier.

6. A composition comprising the extract of claim 4 and an anti-angiogenic or immunostimulatory agent.

7. A method for inhibiting the growth of endothelial cells, comprising delivering to the cells a growth inhibitory amount of the extract of claim 4.

8. A method of inhibiting vascularization in a tissue, comprising delivering to the tissue an anti-vascularization amount of the extract of claim 4.

9. A method of treating a disorder associated with pathological neovascularization in a subject, comprising administering to a subject a therapeutically effective amount of the extract of claim 4.

10. The method of claim 9, wherein the disorder is selected from the group consisting of cancer, arthritic conditions, neovascular-based dermatological conditions, diabetic retinopathy, restinosis, Karposi's Sarcoma, age-related macular degeneration, telangectasia, glaucoma, keloids, corneal graft rejection, wound granularization, angiofibroma, Osler-Webber Syndrome, myocardial angiogenesis, and scleroderma.

11. The method of claim 7 or 8, wherein the disorder is an arthritic condition selected from the group consisting of psoriatic arthritis, rheumatoid arthritis and osteoarthritis.

12. The method of claim 7 or 8, wherein the delivering is selected from the group consisting of orally, intravenously, intraperitoneally, and transdermally.

13. The method of claim 8, wherein the subject is an animal.

14. The method of claim 13, wherein the animal is selected from the group consisting of a pet, a farm animal or a human patient.

15. A method for screening for a therapeutic agent for inhibiting neovascularization or endothelial cell growth comprising the steps of:
a) contacting the agent with a suitable cell or tissue sample;
b) contacting a separate sample of the suitable cell or tissue with a therapeutically effective amount of the extract of claim 4; and
c) comparing the growth of the sample of step (a) with the growth of the sample of step (b), and wherein any agent of step (a) that inhibits the growth to the same or similar extent as the sample of step (b) is a therapeutic agent for inhibiting neovascularization or the growth of endothelial cells.

16. The method of claim 15, wherein the contacting is in vitro or in vivo.

17. The method of any one of claim 7, 8 or 9, further comprising administering or delivering an effective amount of an anti-angiogenic agent or therapy.

18. The method of any one of claim 7, 8 or 9, further comprising delivering or administering an anti-tumor therapy or agent.

19. A kit for treating a disorder associated with pathological neovascularization in a host, comprising a therapeutically effective amount of the extract of claim 4 and instructions for use.

20. The kit of claim 19, wherein the disorder is selected from the group consisting of cancer, arthritic conditions, neovascular-based dermatological conditions, diabetic retinopathy, Karposi's Sarcoma, age-related macular degeneration, restinosis, telangectasia, glaucoma, keloids, corneal graft rejection, wound granularization, angiofibroma, Osler-Webber Syndrome, myocardial angiogenesis, and scleroderma.

21. The kit of claim 20, wherein the disorder is an arthritic condition selected from the group consisting of psoriatic arthritis, rheumatoid arthritis and osteoarthritis.
